(19) United States
(12) Patent Application Publication
(10) Pub. No.: US 2023/0008584 A1
(43) Pub. Date: Jan. 12, 2023

(54) INJECTION DEVICE SUITABLE FOR INJECTING PARTICLES INTO A BONE CAVITY

(71) Applicant: SPINEDUST, Marseille (FR)

(72) Inventors: Olivier LEVRIER, Marseille (FR); Noël GRAZIANI, Marseille (FR)

(21) Appl. No.: 17/784,794

(22) PCT Filed: Dec. 9, 2020
(86) PCT No.: PCT/FR2020/052359

§ 371 Date: Jun. 13, 2022

(30) Foreign Application Priority Data
Dec. 11, 2019 (FR) FR1914143

(51) Int. Cl. A61B 17/88

(52) U.S. Cl. CPC A61B 17/8819; A61B 17/8811; A61B 17/8822; A61B 17/8855

(57) ABSTRACT

An injection device for injecting particles into a bone cavity includes a reservoir, an injection needle having a tubular shape around a main axis and a proximal end fixed to a base of the reservoir and open to the reservoir, and a distal end opposite to the proximal end, a worm screw movable in rotation inside the injection needle and extending at least from the proximal end up to at least the distal end of the injection needle, and an actuator coupled to the worm screw to drive it in rotation.

### TECHNICAL FIELD

The disclosure concerns the field of orthopedics and relates to an injection device for injecting particles into a bone cavity, as well as an injection kit comprising such an injection device as well as related particles.

### BACKGROUND

Percutaneous vertebroplasty is a surgical technique, minimally invasive and guided by X-ray imaging, which consists in injecting into a vertebra, by means of a bone biopsy needle and from the pedicle, a bone cement composition which will harden in order to treat a fracture (for example in the context of a tumor lesion fracture, aggressive angiomas or osteoporotic vertebral fractures).

Bone cement compositions based on poly(methyl methylacrylate) (hereinafter abbreviated as “PM MA”) are usually used for this type of surgery.

However, PMMA-based bone cement compositions have the following drawbacks:
the polymerization of PMMA is a significant exothermic reaction which can damage the surrounding organic tissues once the injection of bone cement composition has been carried out;
the mechanical properties of PMMA being far from those of bone, the bone cement may damage it if the bone cement is too rigid, in particular in the case of osteopenic bone;
PMMA-based bone cement compositions do not have in vivo activity for bone bonding. In other words, such compositions cannot form a chemical bond with human bone tissues or cannot be replaced by newly formed bones. Therefore, the interface between such bone cement compositions and bone may be disrupted over time, causing a risk of bone loosening.

This is why, in view of these drawbacks, the development of alternatives to PMMA-based bone cement compositions would be entirely beneficial for practitioners and patients.

### SUMMARY

The inventor of the present application has developed an injection device which makes it possible to inject particles into a bone cavity, which perfectly overcomes the drawbacks associated with PMMA-based bone cement compositions that have been mentioned hereinabove.

The disclosure also relates to an injection device adapted for an injection of particles into a bone cavity, said injection device comprising:
a reservoir capable of containing at least in part the particles;
an injection needle having a tubular shape around a main axis and provided with a proximal end fixed to a base of the reservoir and open to the reservoir, and an distal end opposite to the open proximal end; and
a worm screw movable in rotation around the main axis inside the injection needle and extending at least from the proximal end up to at least the distal end of the injection needle;
an actuator coupled to the worm screw to drive it in rotation.

Such an injection device is particularly adapted to inject the particles through the injection needle by being conveyed by the worm screw (also known as the Archimedes screw). The use of the worm screw promotes the displacement of the particles, while limiting the risks of aggregation of the particles which would then form a blockage harmful to the injection. Once injected, these particles will be distributed in the bone cavity and will provide high mechanical strength. Unlike PMMA-based bone cement compositions which are non-porous, the particles will form a porous and granular material which thus has a lighter, more elastic and more resistant structure. Furthermore, this particle-based material integrates perfectly within the bone, since the vertebrae are porous materials.

According to one possibility, the actuator comprises a shaft coupled in rotation to the worm screw, said shaft emerging from the reservoir to be engaged with a motor.

According to another possibility, a piston is slidably mounted in the reservoir and movable in translation according to the main axis. Such a piston is not intended to push the particles (or a bone cement composition comprising such particles dissolved in a liquid or gel composition), insofar as the injection is carried out by the rotating worm screw. Indeed, it is preferable to avoid applying strong pressure on the upper portion of the reservoir, which would cause an accumulation of particles.

Advantageously, the injection device comprises at least one dispersion means capable of dispersing and suspending the particles in a liquid or gel composition, for example of the biological glue or polymeric gel type, inside the reservoir. The dispersing means is/are intended to fluidize the bone cement composition (which comprises the particles dissolved in the liquid or gel composition) for smooth flow through the injection needle, by dispersing the particles to avoid aggregations and sedimentation of these particles.

In a particular embodiment, the dispersing means is selected from the following means or a combination of all or part of the following means:
an introduction system for introducing the liquid or gel composition inside the reservoir, said introduction system comprising a circulation means located outside the reservoir and ensuring a circulation of the liquid or gel composition in one or several introduction conduits connected to one or several introduction points opening into the base of the reservoir:
a vibrator system to apply a vibration to the reservoir or to the injection needle;
a stirring system for stirring the particles dissolved in the liquid or gel composition inside the reservoir:
an oscillating system comprising a piston mounted to slide in the reservoir and movable in translation according to the main axis, and an oscillating actuator capable of imparting an oscillating movement to the piston according to the main axis.

The introduction system makes it possible to introduce the liquid or gel composition at the level of the base (in the lower portion of the reservoir), thus generating a liquid flow capable of detaching the particles from the base and of dispersing the particles at the level of the proximal end of the injection needle, and therefore at the input to the worm screw.

This introduction system may be supplemented with a recirculation system for a recirculation of the liquid or gel composition, which would include one or several evacuation points opening into the reservoir (preferably in the upper portion of the reservoir) and connected to one or several evacuation conduits for a return of the liquid or gel composition at the level of the circulation means. Thus, the liquid or gel composition entering at the level of the introduction point(s), and the liquid or gel composition exiting at the level of the evacuation point(s), the circulation means (such as for example a pump) ensuring the circulation of the liquid or gel composition between the evacuation point(s) and the introduction point(s).

The vibrator system applies a vibration which makes it possible to break up the particle clusters, in the reservoir and/or in the injection needle (and therefore inside the worm screw). The vibrator system applies for example a vibration at a frequency comprised between 0.5 Hz and 10 KHz. The vibrator system is for example a mechanical system connected to a motor and in contact with the reservoir and/or the injection needle.

The stirring system applies a mechanical stirring movement inside the reservoir, and it may comprise a stirrer (such as an agitator or a paddle) engaged to a motor located outside the reservoir. This stirring system thus makes it possible to break up the particle clusters and therefore to fluidize the bone cement com position.

The oscillating system applies an oscillating movement to the piston according to the main axis, which amounts to an oscillating movement from bottom to top in the upper portion of the reservoir, which provides a pressure gradient that contributes to a dispersion of the particles inside the reservoir.

In a particular embodiment, the worm screw has a generally cylindrical shape or a generally frustoconical shape. The use of a frustoconical worm screw decreases the local volume of the particles (if any of the bone cement composition) and forces the particles to reorganize, thus increasing the split ratio in the granular medium. In granular rheology, a higher split ratio leads to a higher friction coefficient between the particles and the solid stress. However, a cylindrical worm screw limits the split ratio in the granular medium.

According to one possibility, the worm screw extends entirely within the injection needle, by extending from the proximal end up to the distal end of the injection needle.

According to another possibility, the worm extends inside the injection needle and also inside the reservoir, by extending beyond the proximal end of the injection needle, so that the worm screw has at least two successive portions comprising:
a lower portion which extends from the proximal end up to the distal end of the injection needle;
an upper portion which prolongs the lower portion beyond the proximal end and which extends inside the reservoir.

In an advantageous embodiment, the worm screw is adjustable in translation along the main axis, with the advantageous aim of allowing impaction of the particles inside the bone cavity by means of the distal end of the worm screw.

Such a possibility of adjustment thus makes it possible to retract the worm screw inside the injection needle, that is to say without protruding from the distal end of the injection needle, and to deploy the worm screw out of the injection needle, protruding beyond the distal end of the injection needle so as to be immersed inside the bone cavity, in order to promote impaction of the particles inside the bone cavity. The progression of the worm screw in the bone cavity, and therefore out of the injection needle, may advantageously be radiologically controlled, and a locking of the optimal deployment position of the worm screw will be carried out.

In a particular embodiment, the injection device further comprises an expandable implant comprising at least one catheter and an expandable envelope configurable between:
a contracted state in which the expandable envelope is radially constrained in the catheter for delivery of the expandable envelope within the bone cavity, and
a deployed state in which the expandable envelope is extended out of the catheter with a bag-like configuration to be filled with particles.

Such an expandable envelope is thus provided to be deployed inside the bone cavity, and thus to form a containment bag inside the bone cavity, and then the particles (alone or dissolved in a liquid or gel composition) will be injected inside this expandable envelope which will provide containment of the particles. Such an expandable envelope will also make it possible to avoid a secondary migration of the particles into the venous blood circulation. The use of such an expandable envelope optionally makes it possible to dispense with a liquid or gel composition and to inject the particles directly and solely; and in this case it is this expandable envelope which will provide the containment function.

According to one possibility, the expandable envelope includes at least a plurality of threads arranged to form together a mesh, or the expandable envelope is a solid envelope made of an elastic or inflatable material.

According to another possibility, the catheter passes through the worm screw; the worm screw then being provided with an elongated orifice in its length, through which the catheter is inserted to reach the bone cavity and then deploy the expandable envelope inside the bone cavity.

The disclosure also relates to an injection kit which comprises at least:
an injection device as described hereinabove; and
particles adapted to be injected inside a bone cavity by means of said injection device.

Advantageously, this injection kit further comprises a bone vibrator capable of applying a vibration to a bone during injection of the particles inside a bone cavity of the bone. Thus, this bone vibrator will apply a vibration to the bone, thus avoiding an aggregation of the particles inside the bone cavity which would lead to the formation of empty spaces and therefore to an undesirable partial filling

Even more advantageously, the worm screw has a screw pitch greater than or equal to a maximum size of the particles of the bone cement composition.

Advantageously, the particles are selected from particles of hydroxyapatite, poly(lactic-glycolic)acid (hereinafter abbreviated as PLGA), starch or chitosan.

In a particular embodiment, the particles are dissolved in a liquid or gel composition to form together a bone cement composition, wherein said liquid or gel composition includes a liquid or gel solvent adapted to diffuse through the bone.

The liquid or gel composition has its solvent which is in a liquid or gel state before the injection of the bone cement composition, and the particles are homogeneously distributed therein. When the bone cement composition has been injected into a bone cavity, the solvent contained in the liquid or gel composition will diffuse into the patient's body. Due to the diffusion of the solvent, this will induce a densification of the particles which will be distributed homogeneously inside the bone cavity.

According to a first possibility, the liquid or gel composition is a polymeric gel comprising a polymer dissolved in a solvent, said polymer being selected from:
polyethylene glycol, hyaluronic acid or hydroxypropyl methylcellulose dissolved in water;
ethylene vinyl alcohol or cellulose acetate dissolved in dimethyl sulfoxide;
ethyl cellulose dissolved in ethanol.

According to a second possibility, the liquid or gel composition is a biological glue which is selected from biological glues based on:
fibrinogen or thrombin, fibrinogen and thrombin being optionally in association with aprotinin,
autologous fibrin,
collagen, or
N-butyl-cyanoacrylate.

The biological glue and the polymeric gel described above each contain a solvent capable of diffusing through the bone. They are in a liquid state before the injection of the bone cement composition and additionally contain the particles which are homogeneously distributed therein. When the bone cement composition has been injected into a bone cavity, the solvent contained in the biological glue or the polymeric gel will diffuse into the patient's body. Due to the diffusion of the solvent, this will induce the solidification of the gel or the biological glue within which the particles will be trapped and distributed therein in a homogeneous manner.

The bone cement composition, based on the biological glue or the polymeric gel described hereinabove, thus has the following advantages:
being in the liquid state before its injection, which facilitates its injection and allows a homogeneous distribution of the particles;
the particles are brought inside the bone and remain distributed therein in a homogeneous manner thanks to their trapping within the gel or the biological glue which has solidified due to the diffusion of the solvent in the patient's body.

The solidification within the bone of the polymeric gel or the biological glue trapping the particles makes it possible to obtain a material whose mechanical properties are close to those of the patient's bone. These mechanical properties are better than those of PMMA-based bone cement compositions. Indeed, the particles contained in the bone cement composition contribute to increasing the mechanical strength of said composition. This is why the mechanical strength of the bone cement composition is greater than that of the PMMA-based composition.

Unlike the PMMA-based bone cement compositions which are non-porous, the bone cement composition according to the disclosure has the advantage of obtaining, after its injection and the solidification of the polymeric gel or the biological glue, a porous and granular material which thus presents a lighter, more elastic and more resistant structure. Furthermore, this material integrates perfectly within the bone, since the vertebrae are porous materials.

The similarity of the mechanical properties with those of the bone of the porous and granular material thus obtained from the injection of the bone cement composition according to the disclosure also has the advantage of making said injection less traumatic.

The bone cement composition according to the disclosure also has the advantage of inducing the formation in vivo of bone cells and therefore the formation of bone.

Unlike PMMA-based bone cement compositions, there is no in vivo polymerization of said bone cement composition according to the disclosure. This avoids all the problems related to the exothermic reactions of the polymerization which have been mentioned hereinabove. Indeed, in the bone cement composition according to the disclosure, all of its constituents are chemically stable before being injected into the patient's bone. There is therefore no risk of a damaging chemical reaction within the bone.

The particles may be spherical or irregular in shape.

Regarding the hydroxyapatite particles, their size may widely vary. For example, their size may be comprised between 15 and 45 μm or between 15 and 125 μm or between 25 and 75 μm or between 70 and 210 μm or between 32 and 106 μm or between 45 and 130 μm or between 160 and 400 μm or between 400 and 1000 μm or between 1000 and 2000 μm. The particles may be a mixture of particles whose size may be selected from at least two of these size ranges. For example, the particles may be a mixture of particles of different sizes, and for example of sizes comprised between 15 and 125 μm, between 160 and 400 μm and between 400 and 1000 μm.

Regarding the PLGA particles, their size may be comprised between 5 and 250 μm. Optionally, the PLGA particles encapsulate at least one active substance. It may be an antibiotic or antitumor active substance.

Regarding the starch particles, their size may be comprised between 50 and 200 μm.

In general, the particles advantageously have a size of less than 2000 μm.

Advantageously, the liquid or gel composition comprises a radiopaque agent.

The radiopaque agent may be a micronized metal powder. It may be a powder of tantalum, tungsten, mineral iodine, barium or bismuth. Alternatively, the radiopaque agent may be a gadolinium solution of ethylenediaminetetraacetic acid.

The radiopaque agent may also be an ethiodized oil.

The radiopaque agent makes it possible to visualize in real time the location of the injection of the bone cement composition within the bone according to the disclosure.

In the bone cement composition, the ratio of the mass percentage of the liquid or gel composition (polymeric gel or biological glue) to the mass percentage of particles is at most 1:4. This thus makes it possible to obtain a paste which can be injected into the bone by the injection device according to the disclosure as previously described. The paste may be more or less dense in particles. Thus, if said ratio is between 1:4 and 1:9, the paste of bone cement composition is in the form of a low-density aggregate. If said ratio is between 1:9 and 1:99, the bone cement composition paste is in the form of a dense aggregate.

This ratio may easily be adapted according to the age and state of health of the patient into whom the bone cement composition according to the disclosure is injected. Indeed, this ratio will be different depending on whether the patient is a person with osteopenia or osteoporosis or a person with a vertebral fracture. Due to its essential constituents, which are on the one hand a polymeric gel or a biological glue and on the other hand particles, the bone cement composition according to the disclosure thus has the advantage of being adaptable according to the specific needs of the patient into whom said composition is injected. Depending on the patients, a more or less dense bone cement composition according to the disclosure will be injected.

The bone cement composition may further comprise fibers. The length of the fibers may be comprised between 5 μm and 5 mm. The ratio between the length of the fibers and their average diameter may be comprised between 2 and 1000. The fibers make it possible to reinforce the material resulting from the solidification of the polymeric gel or the biological glue trapping the particles.

These fibers may for example be fibers of natural origin, and in particular of animal origin such as silk fibers or of plant origin such as cellulose fibers. These fibers may alternatively be synthetic fibers, such as for example chitosan fibers, polylactic acid fibers (also abbreviated as PLA), poly(lactic-glycolic)acid fibers (also abbreviated as PLGA), fibers of carbon. In another variant, these fibers may be a mixture of fibers, and for example a mixture of fibers of natural origin and synthetic fibers.

### BRIEF DESCRIPTION OF THE DRAWINGS

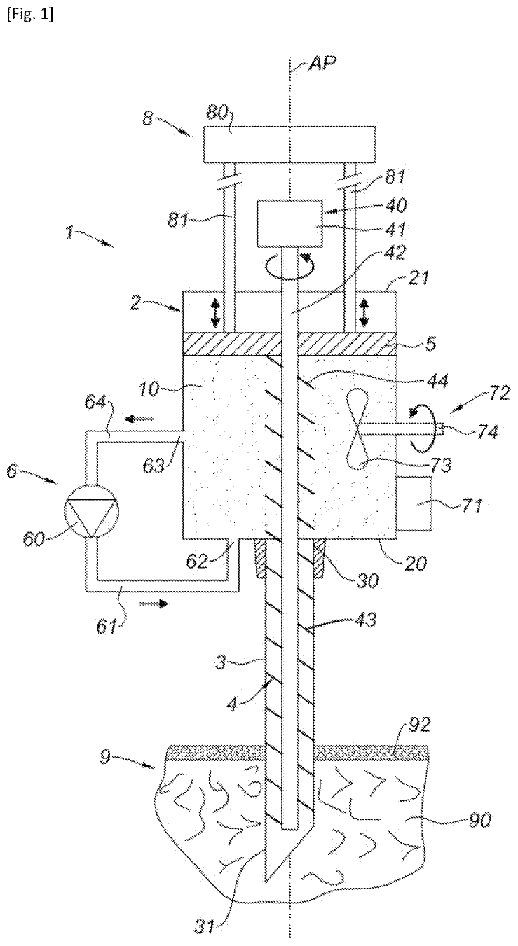

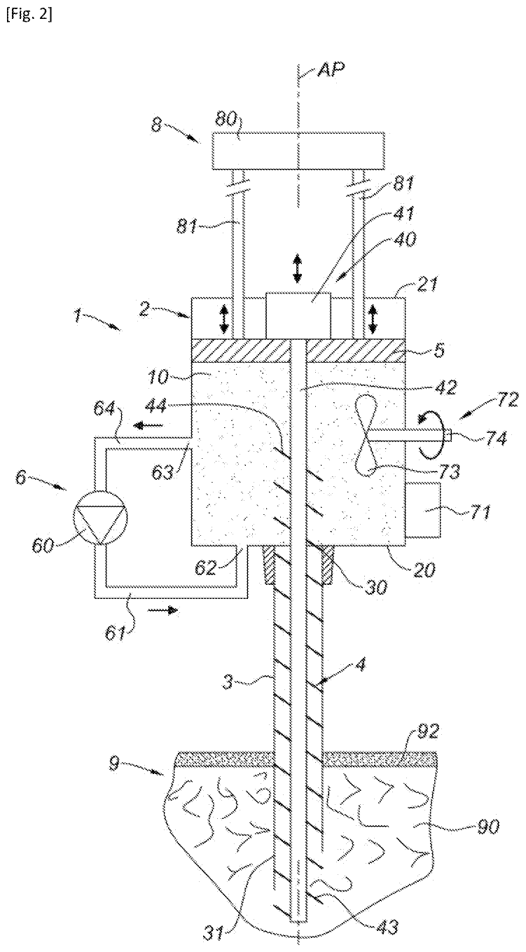

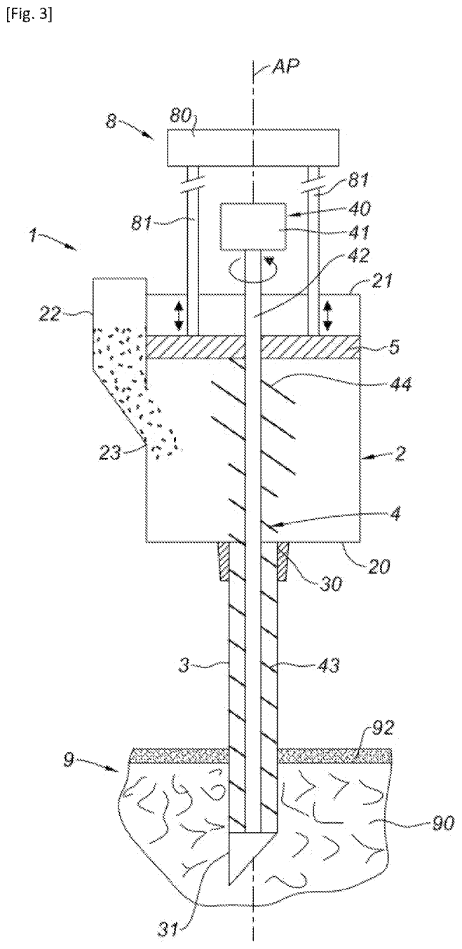

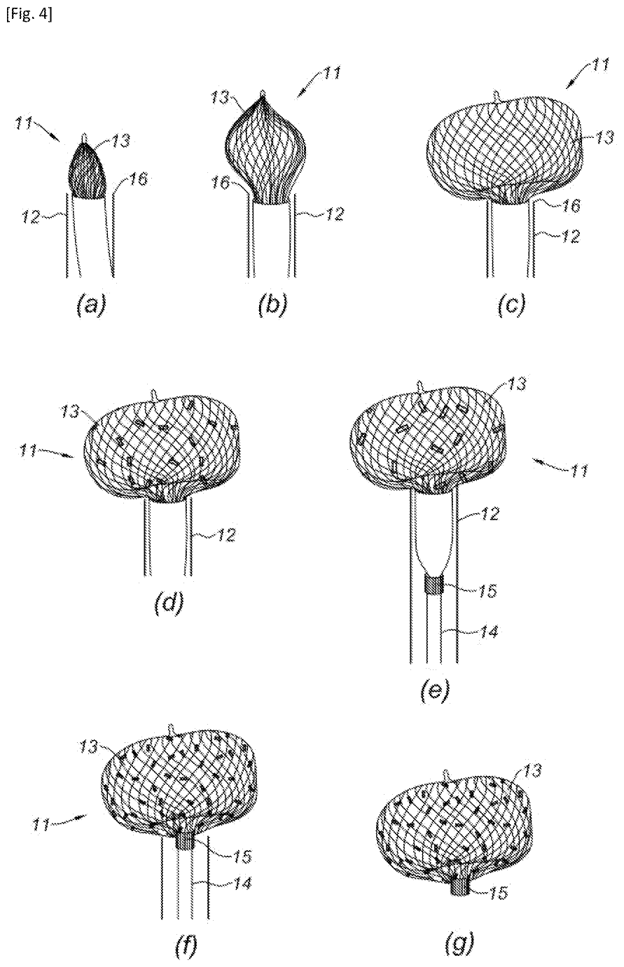

The disclosure will be better understood with the aid of the detailed description which is disclosed hereinbelow with reference to the appended figures representing, as a non-limiting example, several embodiments of an injection device according to disclosure, as well as an illustration of an expandable implant adapted to the disclosure.

[FIG. 1] is a schematic illustration of an injection kit according to a first embodiment of the disclosure, comprising a first injection device and a bone cement composition, wherein the first injection device is in a first configuration;

[FIG. 2] is a schematic illustration of the injection kit of FIG. 1, wherein the first injection device is in a second configuration;

[FIG. 3] is a schematic illustration of an injection kit according to a second embodiment of the disclosure, comprising a second injection device and a bone cement composition; and

[FIG. 4] is a schematic illustration of an expandable implant adapted to the disclosure, during successive steps (a) to (g) of inserting its expandable envelope inside a bone cavity.

### DETAILED DESCRIPTION OF THE DRAWINGS

Referring to FIGS. 1 to 3, an injection kit comprises particles and an injection device 1 adapted to inject the particles inside a bone cavity 90 of a bone 9. For the Following description, with regard to the injection device 1, the same numerical references will be used to refer to components or elements that are identical, similar or having identical functions.

In an advantageous embodiment, the particles are dissolved in a liquid or gel composition to form together a bone cement composition 10, where this liquid or gel composition includes a liquid or gel solvent adapted to diffuse through the bone 9.

The bone cement composition 10 therefore comprises at least the particles dissolved in the liquid or gel composition which is a biological glue or a polymeric gel, with a ratio of the mass percentage of the polymeric gel or of the biological glue to the mass percentage of particles which is at most 1:4.

The particles are selected from particles of hydroxyapatite, poly(lactic-glycolic)acid (hereinafter abbreviated as PLGA), starch or chitosan.

The polymer gel comprises a polymer dissolved in a solvent, where the polymer is selected from:
polyethylene glycol, hyaluronic acid or hydroxypropyl methylcellulose dissolved in water;
ethylene vinyl alcohol or cellulose acetate dissolved in dimethyl sulfoxide;
ethyl cellulose dissolved in ethanol.

The biological glue is selected from biological glues based on:
fibrinogen or thrombin, fibrinogen and thrombin being optionally in association with aprotinin,
autologous fibrin,
collagen or
N-butyl-cyanoacrylate.

The bone cement composition 10 optionally comprises a radiopaque agent such as a micronized metal powder selected from tantalum, tungsten, barium, bismuth, or mineral iodine powders. Alternatively, the radiopaque agent may be a gadolinium solution of ethylenediaminetetraacetic acid or an ethiodized oil.

The following description relates to the injection device 1, whether in the first embodiment illustrated in FIGS. 1 and 2 or in the second embodiment illustrated in FIG. 3. The injection device 1 comprises a reservoir 2 capable of containing the bone cement composition 10, where this reservoir 2 has a base 20 (forming a bottom wall) in the lower portion and has a top 21 (open or closed by a removable lid or not) in the upper portion. The reservoir 2 may has a generally cylindrical shape.

In the example of FIG. 3, the injection device 1 also comprises an attached reservoir 22 which is fixed to the reservoir 2 and which receives particles internally, and in particular bulk particles, where these particles are those used to form the bone cement composition 10. This attached reservoir 22 is in communication with the reservoir 2 through an opening 23, and for example an opening provided with a configurable flap between an open position (for an opening of the communication) and a closed position (for a closing of the communication). Thus, the particles may be introduced gradually into the reservoir 2. It could be considered that this reservoir 2 is filled beforehand (and possibly continuously supplied) with biological glue or polymeric gel, just as it could be considered that this reservoir 2 only contains the dry particles in the case where the particles are introduced without a liquid or gel composition.

The injection device 1 comprises an injection needle 3 of tubular shape around a main axis AP and provided with a proximal end 30 fixed to the base 20 of the reservoir 2 (for example by means of a connection socket) and open to the reservoir 20, and an open distal end 31 opposite the proximal end 30. The proximal end 30 is thus in fluid connection with the interior of the reservoir 20, and the distal end 31 is provided to penetrate inside the bone cavity 90. The distal end 31 may be provided with a pointed or beveled tip, to pierce the compact and hard outer layer 92 of the bone 9; optionally after pre-piercing of this outer layer 92.

The injection device 1 comprises a worm screw 4 movable in rotation around the main axis AP inside the injection needle 3 and extending at least from the proximal end 30 up to at least the distal end 31 of the injection needle 3. This worm screw 4 is thus mounted inside an inner channel of the injection needle 3; this inner channel having a generally cylindrical shape. The worm screw 4 has a generally cylindrical shape (as illustrated in FIG. 1) or a generally tapered shape (in a non-illustrated variant). This worm screw 4 has a maximum outer diameter which is substantially less than an inner diameter of the inner channel of the injection needle 3, for mounting almost without a clearance of the worm screw 4 in the inner channel of the injection needle 3; a clearance being provided to allow the worm screw 4 to rotate inside the inner channel of the injection needle 3.

The worm screw 4 has a screw pitch greater than or equal to a maximum particle size of the bone cement composition 10, and for example a screw pitch equal to or greater than 2 millimeters.

In a not illustrated embodiment, the worm screw 4 extends from the proximal end 30 up to the distal end 31 of the injection needle 3, and thus has a length (distance measured along the axis of rotation of the worm screw 4) substantially equivalent to the length of the injection needle 3.

In the embodiments of FIGS. 1 and 2 and FIG. 3, the worm screw 4 extends from the proximal end 30 up to the distal end 31 of the injection needle 3, and also extends beyond the proximal end 30 such that this worm screw 4 extends in part inside the reservoir 2. In other words, the worm screw 4 has two successive portions:
a lower portion 43 which extends from the proximal end 30 up to the distal end 31 of the injection needle 3, and thus has a length substantially equivalent to the length of the injection needle 3;
an upper portion 44 which prolongs the lower portion 43 and which extends inside the reservoir 2.

Moreover, the worm screw 4 is adjustable in translation along the main axis AP, and this worm screw 4 is thus configurable between:
a first configuration, visible in FIG. 1, in which the worm screw 4 is retracted inside the injection needle 3, in the way that this worm screw 4 does not protrude from the distal end 31 of the injection needle 3; and
a second configuration, visible in FIG. 2, in which the worm screw 4 is deployed out of the injection needle 3, in the way that this worm screw 4 protrudes from the distal end 31 of the injection needle 3 to be immersed inside the bone cavity 90.

Preferably, this adjustment is controlled in the way that the projecting length of the worm screw 4, beyond the distal end 31 of the injection needle 3, is controlled and lockable, which means that the surgeon may adjust and block this projecting length.

Although not shown, it also could be considered that the worm screw 4 of FIG. 3 is also adjustable in translation along the main axis AP.

The worm screw 4 has the function of guiding and conveying the particles of the bone cement composition 10 from the reservoir 2 up to the distal end 31 of the injection needle 3, and therefore up to the bone cavity 90 of the bone 9. In the example of FIG. 1, this guiding and this conveying of the particles by the worm screw 4 take place from the proximal end 30 of the worm needle 3, therefore from the base 20. In the example of FIG. 2, this guiding and this conveying of the particles by the worm screw 4 take place from inside the reservoir 2, by means of the upper portion 44 of the worm screw 4 which begins to convey the particles while providing stirring due to the rotation of this upper portion 44 in the reservoir 2 which generates a swirling flow.

In the embodiment of FIG. 3, the upper portion 44 of the worm screw 40 may have a diameter greater than that of the lower portion 43, and it is in particular possible that this upper portion 44 has an increasing diameter from the lower portion 43. In the embodiment of FIGS. 1 and 2, the upper portion 44 of the worm screw 40 has the same diameter as the lower portion 43.

The injection device 1 comprises an actuator 40 coupled to worm screw 4 to drive it in rotation. In the non-limiting example illustrated in FIG. 1, the actuator 40 comprises a motor 41 engaged with a shaft 42, where this shaft 42 is coupled in rotation to the worm screw 4, and in particular the shaft 42 has a lower portion around which the worm screw 4 is fixed. The shaft 42 comes out of the reservoir 2 to be engaged with the motor 41 placed outside the reservoir 2.

In the case where the worm screw 4 is adjustable in translation along the main axis AP, then the actuator 40, and therefore the motor 41 and the shaft 42, are secured in translation to the worm screw 4.

The injection device 1 comprises a piston 5 which is slidably mounted in the reservoir 2 and movable in translation according to the main axis AP. This piston 5 is crossed in a sealed manner by the shaft 42.

It may be considered that this piston 5 is screwed on the periphery of the shaft 42, which allows an advance of the piston 5 in proportion to the volume evacuated by the worm screw 4 inside the bone cavity 90. In this case, the piston 5 must be able to rotate and thus the actuator 40 must be adapted so that the piston 5 can move in a spiral.

The injection device 1 also comprises one or several dispersion means capable of dispersing and suspending the particles of the bone cement composition 10 in the biological glue or the polymeric gel inside the reservoir 2.

A first dispersion means is an introduction system 6 for introducing a biological glue or polymeric gel inside the reservoir 2, where this introduction system 6 comprises a circulation means 60 (like for example a fluidic pump) located outside the reservoir 2 and providing circulation of biological glue or polymeric gel in one or several introduction conduits 61 connected to one or several introduction points 62 opening into the base 20 of the reservoir 2.

This introduction system 6 may for example be supplemented with a recirculation system for recirculation of the biological glue or the polymer gel, which comprises one or several evacuation points 63 opening into the reservoir (preferably in the upper portion of the reservoir2) and connected to one or several evacuation conduits 64 for a return of the biological glue or the polymeric gel at the level of the circulation means 60. Thus, the biological glue or the polymeric gel enters at the level of the introduction point(s) 62, and the biological glue where the polymeric gel comes out at the level of the evacuation point(s) 63, the circulation means 60 ensuring the circulation of the biological glue or of the polymeric gel in the introduction conduit(s) 61 and in the evacuation conduit(s) 64, between the evacuation point(s) 63 and the introduction point(s) 62. The evacuation point(s) 63 and the introduction point(s) 62 may be provided with filters to prevent the passage of the particles inside the conduits 63, 64.

A second dispersion means is a vibrator system 71 for applying a vibration to the reservoir 2 or to the injection needle 3, such as for example a vibration at a frequency comprised between 0.5 Hz and 10 KHz.

A third dispersion means is a stirring system 72 for stirring the bone cement composition 10 inside the reservoir 2, and it may comprise a stirrer 73 engaged with a motor (not illustrated) located outside the reservoir 2 via a connecting shaft 74 which passes through a wall of the reservoir 2 or which passes through the piston 5.

A fourth dispersion means is an oscillating system 8 comprising the piston 5 and comprising an oscillating actuator 80 capable of imparting an oscillating movement to the piston 5 according to the main axis via one or several transmission elements 81 which transmit an oscillating movement generated by the oscillating actuator 90 to the piston 5.

Referring to FIG. 4, it may be considered to provide that the injection device 1 further comprises an expandable implant 11 comprising at least one catheter 12 and an expandable envelope 13 configurable between:
a contracted state (visible on FIG. 4(a)) in which the expandable envelope 13 is radially constrained in the catheter 12 for a delivery of the expandable envelope 13 within the bone cavity, and
an expanded state (visible in FIGS. 4(c) to 4(g)) in which the expandable envelope 13 is extended out of the catheter 12 with a bag-like configuration.

This expandable implant 11 thus comprises a tubular thrust member 14 (visible in FIGS. 4(e) and 4(f)) formed of a tubular rod sliding inside the catheter 12 and connected by a ring 15 to the expandable envelope 13, so that the particles (visible in FIGS. 4(d) to 4(g)) can be injected inside the expandable envelope 13 through this tubular thrust member 14.

The catheter 12 is provided with a proximal end 16 which opens into the bone cavity, and the expandable envelope 13 is deployed while exiting this proximal end 16.

Although not illustrated, the catheter 12 passes through the worm screw 4, and therefore crosses the shaft 42, along the main axis AP, and the tubular thrust member 14 has an opening in communication with the reservoir 2 to allow the injection of the particles inside the expandable envelope 13 while passing inside the tubular thrust member 14.

In a first embodiment, the expandable envelope 13 is a self-expanding mesh or grating envelope (which deploys naturally once exiting the catheter 12) which includes at least a plurality of threads arranged to form together a mesh or lattice. In this first embodiment, the mesh has openings (or intermesh spaces), between the threads, the dimensions of which are smaller than the sizes of the particles such that these particles cannot escape from the expandable envelope 13.

In a second embodiment, the expandable envelope 13 is a solid envelope (not meshed) made of a material that is both biocompatible and elastic or inflatable material; it being noted that this material may be resorbable or non-resorbable. In this second embodiment, the expandable envelope 13 is inflated by injection of the liquid or gel composition (such as the previously described biological glue or polymeric gel) via the interior of the tubular thrust member 14. The injection pressure of the liquid or gel composition will contribute to deploying the expandable envelope 13 inside the bone cavity 90. Once the expandable envelope 13 has been inflated, the particles are injected inside the expandable envelope 13 by passing also inside the tubular thrust member 14.

Once the expandable envelope 13 is filled with particles (as seen in FIG. 4(f)), the ring 16 is closed or blocked, and the ring 16 is detached from the tubular thrust member 14, thus making it possible to withdraw both the catheter 12 and the tubular pushing member 14, to leave in the bone cavity the expandable envelope 13 filled with particles and closed by the ring 16 (as visible in FIG. 4(g)).

1. An injection device adapted for injecting particles into a bone cavity, said injection device comprising:
a reservoir capable of containing at least in part the particles;
an injection needle having a tubular shape around a main axis and provided with a proximal end fixed to a base of the reservoir and open to the reservoir, and an distal end opposite to the open proximal end;
a worm screw movable in rotation around the main axis inside the injection needle and extending at least from the proximal end up to at least the distal end of the injection needle; and
an actuator coupled to the worm screw to drive the worm screw in rotation.

2. The injection device according to claim 1, wherein the actuator comprises a shaft coupled in rotation to the worm screw, said shaft emerging from the reservoir to be engaged with a motor.

3. The injection device according to claim 1, wherein a piston is slidably mounted in the reservoir and is movable in translation according to the main axis.

4. The injection device according to claim 1, comprising at least one dispersion means capable of dispersing and suspending the particles in a liquid or gel composition, inside the reservoir.

5. The injection device according to claim 4, wherein the dispersion means is selected from among the following or a combination of all or part of the following:
an introduction system for introducing the liquid or gel composition inside the reservoir, said introduction system comprising a circulation means located outside the reservoir and ensuring a circulation of the liquid or gel composition in one or several introduction conduits connected to one or several introduction points opening into the base of the reservoir;
a vibrator system for applying a vibration to the reservoir or to the injection needle:
a stirring system for stirring the particles dissolved in the liquid or gel composition inside of the reservoir; and
an oscillating system comprising a piston slidably mounted in the reservoir and movable in translation according to the main axis, and an oscillating actuator capable of imparting an oscillating movement to the piston according to the main axis.

6. The injection device according to claim 1, wherein the worm screw has a generally cylindrical shape or a generally frustoconical shape.

7. The injection device according to claim 1, wherein the worm screw extends beyond the proximal end of the injection needle, such that the worm screw has at least two successive portions comprising:
a lower portion which extends from the proximal end up to the distal end of the injection needle; and
an upper portion which prolongs the lower portion beyond the proximal end and which extends inside the reservoir.

8. The injection device according to claim 1, wherein the worm screw is adjustable in translation along the main axis.

9. The injection device according to claim 1, further comprising an expandable implant comprising at least one catheter and an expandable envelope that may be configured between:
a contracted state in which the expandable envelope is radially constrained in the catheter for delivery of the expandable envelope within the bone cavity (90), and
a deployed state in which the expandable envelope is extended out of the catheter with a bag configuration to be filled with particles.

10. The injection device according to claim 9, wherein the expandable envelope includes at least a plurality of threads arranged to form together a mesh, or the expandable envelope is a solid expandable envelope made of an elastic or inflatable material.

11. The injection device according to claim 9, wherein the catheter passes through the worm screw.

12. An injection kit comprising at least:
an injection device according to claim 1; and
particles adapted to be injected inside a bone cavity by said injection device.

13. The injection kit according to claim 12, further comprising a bone vibrator capable of applying a vibration to a bone during an injection of the bone cement composition into a bone cavity of said bone.

14. The injection kit according to claim 12, wherein the worm screw has a screw pitch greater than or equal to a maximum particle size.

15. The injection kit according to claim 12, wherein the particles are selected from particles of hydroxyapatite, poly(lactic-glycolic)acid, starch or chitosan.

16. The injection kit according to claim 12, wherein the particles are dissolved in a liquid or gel composition to form together a bone cement composition, wherein said liquid or gel composition includes a liquid or gel solvent adapted to diffuse through the bone.

17. The injection kit according to claim 16, wherein the liquid or gel composition is a polymeric gel comprising a polymer dissolved in a solvent, said polymer being selected from:
polyethylene glycol, hyaluronic acid or hydroxypropyl methylcellulose dissolved in water;
ethylene vinyl alcohol or cellulose acetate dissolved in dimethyl sulfoxide; and
ethyl cellulose dissolved in ethanol.

18. The injection kit according to claim 16, wherein the liquid or gel composition is a biological glue which is selected from biological glues based on:
fibrinogen or thrombin, fibrinogen and thrombin being optionally in association with aprotinin,
autologous fibrin,
collagen, or
N-butyl-cyanoacrylate.

19. The injection kit according to claim 16, wherein the liquid or gel composition comprises a radiopaque agent, which is for example an ethiodized oil or a micronized metal powder selected from powders of tantalum, tungsten, barium, bismuth, mineral iodine, or a gadolinium solution of ethylenediaminetetraacetic acid.

20. The injection kit according to claim 16, wherein the ratio of the mass percentage of the liquid or gel composition to the mass percentage of particles is at most 1:4.

21. The injection kit according to claim 16, wherein the bone cement composition further comprises fibers.
